(19) United States
(12) Patent Application Publication
(10) Pub. No.: US 2013/0197658 A1
(43) Pub. Date: Aug. 1, 2013

(54) NASAL PASSAGE STENT

(71) Applicants: Julian Prabhu, Huntington Beach, CA (US); Luan M. Prabhu, Mississagi (CA); Sean C. Prabhu, Mississagi (CA); Andre J. Prabhu, Mississagi (CA); Ralph Rogers, Placentia, CA (US)

(72) Inventors: Julian Prabhu, Huntington Beach, CA (US); Luan M. Prabhu, Mississagi (CA); Sean C. Prabhu, Mississagi (CA); Andre J. Prabhu, Mississagi (CA); Ralph Rogers, Placentia, CA (US)

(21) Appl. No.: 13/739,860

(22) Filed: Jan. 11, 2013

Related U.S. Application Data
(63) Continuation of application No. 12/710,466, filed on Feb. 23, 2010, now abandoned.
Application No. 12/710,466 is a continuation-in-part of application No. 12/131,262, filed on Jun. 2, 2008, now abandoned.

(51) Int. Cl. A61F 2/82

(52) U.S. Cl. CPC A61F 2/82
USPC 623/23.7

(57) ABSTRACT

Embodiments of the invention are directed to nasal stents incorporating a filter and methods of use thereof. In one embodiment, a nasal stent has a stent body having a lumen with the same inner diameter (ID) along the length thereof. In an alternative embodiment, the nasal stent has a stent body having a lumen with a decreasing ID along the length thereof. In any embodiment, the stent is capable of having a compressed configuration and an expanded configuration. The nasal stent may further include a tubular insert housing a retaining mechanism to retain a filter at a proximal end thereof. A support may further fix the filter at a proximal end thereof. When inserted within a nostril of a person, the filter may function to filter particulates from the air when a person breathes in.

### CLAIM OF PRIORITY

This application is a continuation-in-part application of application Ser. No. 12/131,262 filed Jun. 2, 2008 and hereby incorporated by reference.

### FIELD OF INVENTION

Medical devices for nasal passages.

### BACKGROUND OF INVENTION

Various external nasal devices may exist that enhance the appearance of a patient or aid the patient's breathing ability. Generally, patients or users can undergo nasal surgery (internal or external), cosmetic surgery, and/or use breathing strips which may allow the patient or user to breathe easier. Millions of patients worldwide are exposed to environmental pollution (including outdoor and indoor contaminants), naturally-occurring irritants such as pollen and/or infectious microorganisms such as bacteria and viruses, and often resort to over-the-counter medications or surgery to experience relief. The use of medications and surgery can cause enormous financial strain on individuals, insurance companies, hospitals and other persons or entities bearing the burden of costs associated with the same.

As a result, there exists a need to alleviate the problems associated with conventional methods and devices designed to aid breathing ability.

### SUMMARY OF INVENTION

A nasal stent comprising: a collapsible body having a proximal end and a distal end, the collapsible body having a lumen therein; and a filter mechanism positioned (i) within the collapsible body or (ii) outside of the collapsible body is herein disclosed. The collapsible body may taper from the distal end to the proximal end. The filter mechanism may be positioned within the collapsible body and may taper from a distal end to a proximal end thereof. Alternatively, the filter mechanism may be tubular in configuration or may be an elongated rectangular tube. The collapsible body may comprise a mesh-like design from one of stainless steel, nickel-titanium, or a bioabsorbable or non-bioabsorbable polymer. In one embodiment, an abluminal surface of the collapsible body may be coated with a therapeutic agent. The therapeutic agent may be one of an allergenic antigen-specific antibody, an anti-inflammatory, an antihistamine, a steroid, or a vaccine. In another embodiment, the therapeutic agent may be combined with a controlled-release agent. The filter mechanism may have carbon media therein.

In an alternative embodiment, the filter mechanism may be positioned outside the collapsible body and the nasal stent may further comprise: (iii) a hollow insert positioned within the proximal end of the collapsible body; (iv) a hollow reinforcing mechanism positioned within the hollow insert, and (iv) a support engaged with the proximal portion of the body. The collapsible body may comprise a mesh-like design from one of stainless steel, nickel-titanium, or a bioabsorbable or non-bioabsorbable polymer. In one embodiment, an abluminal surface of the collapsible body may be coated with a therapeutic agent. The therapeutic agent may be one of an allergenic antigen-specific antibody, an anti-inflammatory, an antihistamine, a steroid, or a vaccine. In another embodiment, the therapeutic agent may be combined with a controlled-release agent.

### BRIEF DESCRIPTION OF DRAWINGS

FIG. 1A illustrates a side view of a nasal stent according to an embodiment of the invention.

FIG. 1B illustrates a front view of the nasal stent of FIG. 1A.

FIG. 2A illustrates a side view of a stent body according to an embodiment of the invention.

FIG. 2B illustrates a front view of the stent body of FIG. 2A.

FIG. 3A illustrates a side view of a hollow tubular insert according to an embodiment of the invention.

FIG. 3B illustrates a front view of the insert of FIG. 3A.

FIG. 4A illustrates a side view of a hollow retaining mechanism according to an embodiment of the invention.

FIG. 4B illustrates a front view of the retaining mechanism of FIG. 4A.

FIG. 5A illustrates a side view of a filter according to an embodiment of the invention.

FIG. 5B illustrates a front view of the filter of FIG. 5A.

FIG. 6A illustrates a side view of a support according to an embodiment of the invention.

FIG. 6B illustrates a front view of the support of FIG. 6A.

FIG. 7 illustrates a side view of a nasal stent according to an alternative embodiment of the invention.

FIG. 8 illustrates a side view of a filter according to an alternative embodiment of the invention.

FIG. 9 illustrates a side view of a filter according to an alternative embodiment of the invention.

### DETAILED DESCRIPTION

The following detailed description is of the best currently contemplated modes of carrying out the invention. The description is not to be taken in a limiting sense, but is made merely for the purpose of illustrating the general principles of the invention.

Embodiments of the invention are directed to nasal stents incorporating a filter and methods of use thereof. In one embodiment, a nasal stent has a stent body having a lumen with the same inner diameter (ID) along the length thereof. In an alternative embodiment, the nasal stent has a stent body having a lumen with a decreasing ID along the length thereof. In any embodiment, the stent is capable of having a compressed configuration and an expanded configuration. The nasal stent may further include a tubular insert housing a retaining mechanism to retain a filter at a proximal end thereof. A support may further fix the filter at a proximal end thereof. When inserted within a nostril of a person, the filter may function to filter particulates from the air when a person breathes in.

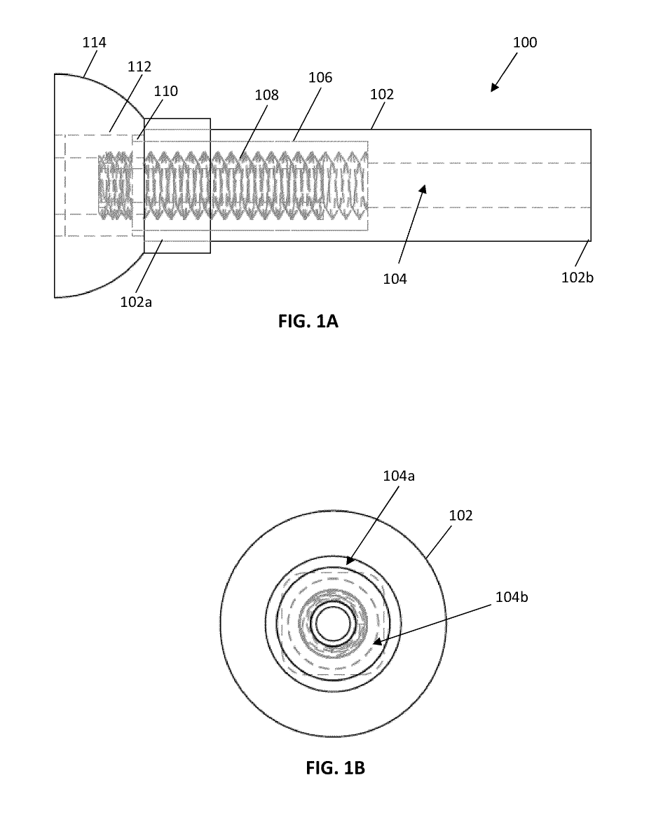

FIG. 1A illustrates a side view of a nasal stent according to an embodiment of the invention. “Stents” are generally tubular-shaped devices which function to hold open a segment of a blood vessel, coronary artery, or other anatomical lumen. As shown, nasal stent 100 comprises a stent body 102 having a proximal end 102a and a distal end 102b. Stent body 102 may be tubular, cylindrical or any other suitable geometric configuration. In the embodiment shown, stent body 102 is tubular in configuration and has a lumen 104 therein. Stent body 102 may be capable of having a compressed configuration and an expanded configuration (explained in more detail below).

In one embodiment, a hollow insert 106 may be positioned within a partial section of lumen 104 at or near the proximal end 102a of stent body 102. The outer diameter (OD) of insert 106 may be smaller than that of lumen 104 and may be expandable. In one embodiment, a hollow retaining mechanism 108 may be positioned within insert 106. Retaining mechanism 108 may be a hollow externally threaded member, such as a hollow flat-head screw, or any other suitable retaining mechanism as known by one of ordinary skill in the art. Retaining mechanism 108 may include a collar 110 positioned about a proximal end of retaining mechanism 108. When retaining mechanism 108 is positioned within insert 106, collar 110 remains outside an opening of proximal end 102a of stent body 102.

In one embodiment, a filter 112 is fitted onto retaining mechanism 108. Filter 112 may include an opening therein (not shown, see FIGS. 5A-5B). In the embodiment in which retaining mechanism 108 is a hollow externally threaded member, the opening of filter 112 may be internally threaded to threadedly engage with retaining mechanism 108. Because of the position of collar 110 on retaining mechanism 108 as discussed previously, filter 112 also remains outside the opening of proximal end 102a of stent body 102. A support 114 may couple to proximal end 102a of stent body 102 (discussed in more detail below). FIG. 1B illustrates a front view of the nasal stent of FIG. 1A.

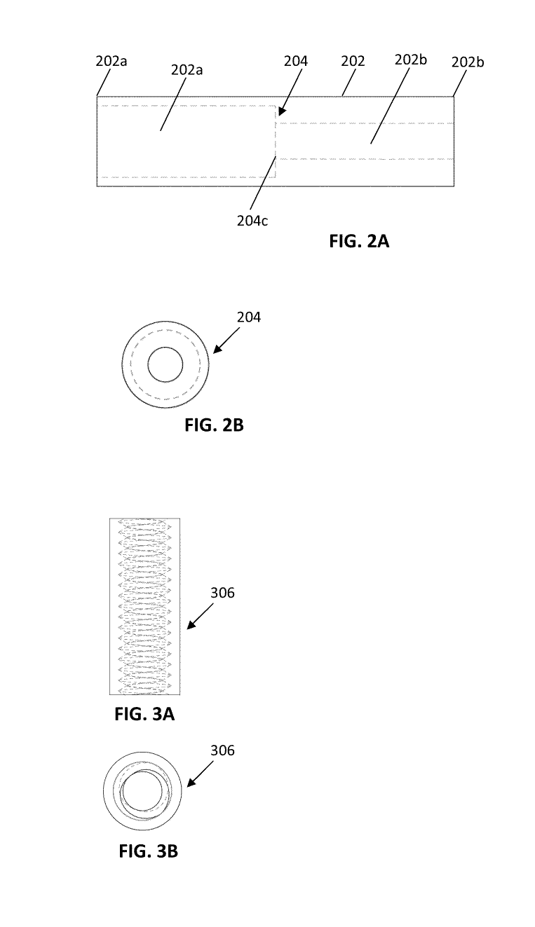

FIG. 2A illustrates a side view of a stent body according to an embodiment of the invention. According to this embodiment, stent body 202 is tubular in configuration. As shown, stent body 202 includes a proximal end 202a and a distal end 202b with a lumen 204 therein. Stent body 202 may be capable of having an expanded configuration 202a and a compressed configuration 202b (explained in more detail below). The inner diameter (ID) of the expanded configuration section 202a may be between about four (4) millimeters and twenty (20) millimeters. The inner diameter (ID) of the compressed configuration 202b may be between about two (2) millimeters and ten (10) millimeters. Stent body 202 may be made of a material including, but not limited to, stainless steel or nickel-titanium mesh or a bioabsorbable or non-bioabsorbable polymer such as those known by one of ordinary skill in the art. FIG. 2B illustrates a front view of the stent body of FIG. 2A.

FIG. 3A illustrates a side view of a hollow insert according to an embodiment of the invention. According to this embodiment, hollow insert 306 is tubular in configuration. Insert 306 may be compressible and/or expandable. The outer diameter (OD) of insert 306 may be smaller than that of an inner diameter (ID) of a stent body (not shown) in which it is positioned therein (see FIGS. 1A-1B). Representatively, insert 306 may have an outer diameter (OD) of between four (4) millimeters and twenty (20) millimeters. In some embodiments, a lumen of insert 306 may be internally threaded to threadedly engage with a retaining mechanism (explained previously). Insert 306 may be press-fitted or secured within the stent body by any suitable means. Insert 306 may be made of a similar material as that of the stent body as explained with reference to FIGS. 2A-2B. FIG. 3B illustrates a front view of the insert of FIG. 3A.

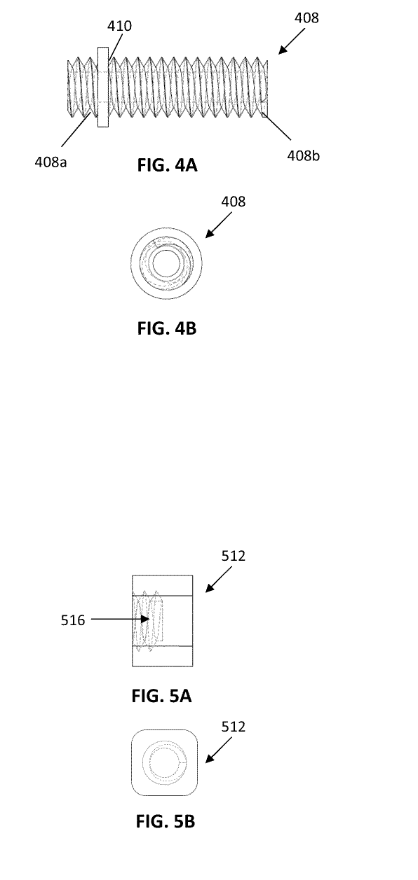

FIG. 4A illustrates a side view of a hollow retaining mechanism according to an embodiment of the invention. According to this embodiment, retaining mechanism 408 is approximately tubular in configuration. In some embodiments, retaining mechanism 408 may be rigid. The outer diameter (OD) of retaining mechanism 408 may be smaller than that of an inner diameter (ID) of an insert (not shown) in which it is positioned therein (see FIGS. 1A-1B). Representatively, retaining mechanism 408 may have an approximate outer diameter (OD) of between four (4) millimeters and twenty (20) millimeters. In one embodiment, retaining mechanism 408 may be positioned within an insert which in turn is positioned within a lumen of a stent body. Retaining mechanism 408 may include a collar 410 positioned about a proximal end 408a of retaining mechanism 408. When retaining mechanism 408 is positioned within the insert, collar 410 remains outside an opening of the stent body.

In some embodiments, an outer circumference of retaining mechanism 408 may be externally threaded to threadedly engage with the insert (explained previously). Retaining mechanism 408 and collar 410 may be made of stainless steel, nickel-titanium or a bioasborbable or non-bioabsorbable polymeric material. FIG. 4B illustrates a front view of the retaining mechanism of FIG. 4A.

FIG. 5A illustrates a side view of a filter according to an embodiment of the invention. Filter 512 may include a partial opening 516 therein. The opening 516 of filter 512 may be internally threaded to threadedly engage with an externally threaded retaining mechanism (see FIGS. 4A-4B). Filter 512 remains outside the opening of a proximal end of a stent body (explained previously). Filter 512 may be, but is not limited to, a carbon or a high efficiency particulate absorbing (HEPA) filter. In one embodiment, filter 512 includes both activated carbon media and a polytetrafluoroethylene (ePTFE) membrane, such as GORE™ Adsorbent Filters available from W. L. Gore & Associates, Inc. Filter 512 may have one or more of the following characteristics: adsorption; high airflow; and liquid resistance. Filter 512 is removable and may be interchanged as often as necessary. FIG. 5B illustrates a front view of the filter of FIG. 5A.

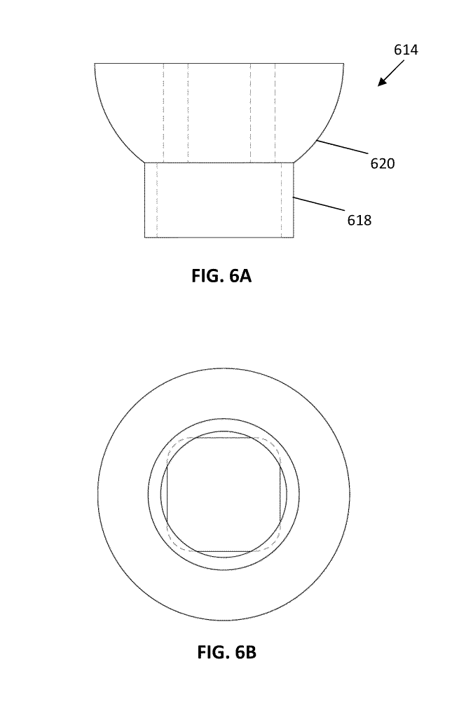

FIG. 6A illustrates a side view of a support according to an embodiment of the invention. Generally, support 614 includes a collar portion 618 and a funnel portion 620. Collar portion 618 is configured to press-fit onto a proximal end of a stent body (see FIGS. 1A-1B). Funnel portion 620 functions both to protect a filter attached to the stent body (see FIGS. 1A-1B) and to retain the nasal stent within the nostril. Support 614 may be made of stainless steel, nickel-titanium or a bioasborbable or non-bioabsorbable polymeric material. FIG. 6B illustrates a front view of the support of FIG. 6A.

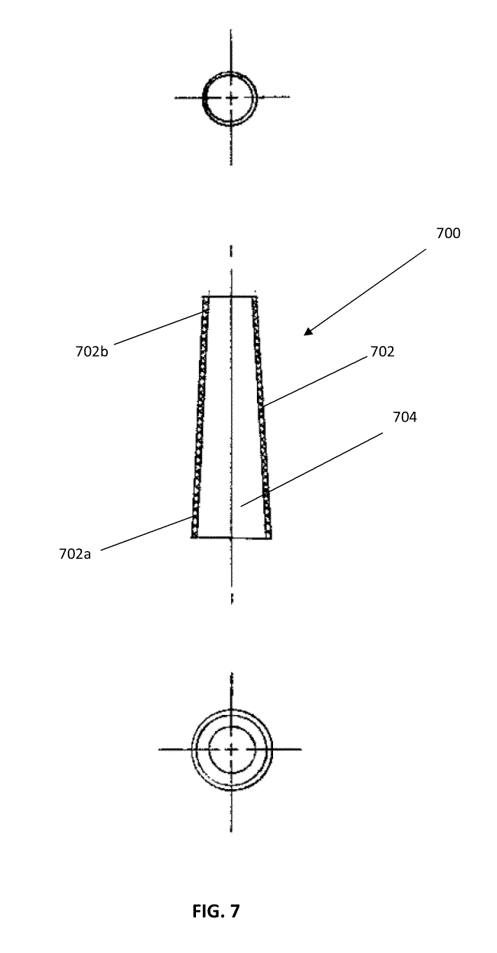

FIG. 7 illustrates a side view of a nasal stent according to an alternative embodiment of the invention. Similar to the previous embodiment, stent 700 may have some or all of the features discussed above; however, according to this embodiment, nasal stent 700 has a stent body 702 having a lumen 704 with a decreasing inner diameter (ID) along the length thereof. That is, the stent body 702 may have a tapered configuration from a distal end 702b to a proximal end 702a. According to some embodiments, the stent may be encapsulated with a bioasborbable or non-bioabsorbable polymeric material to protect nasal tissue growth into the stent.

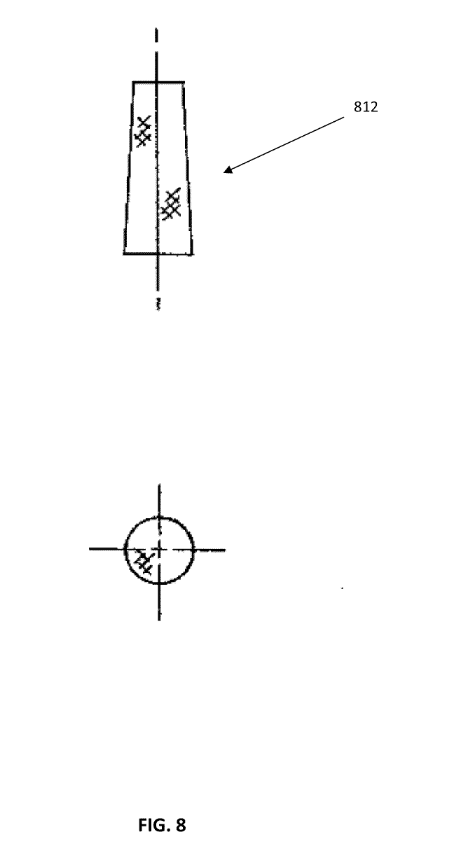

FIG. 8 illustrates a side view of a filter according to an alternative embodiment of the invention. Similar to the previous embodiment, filter 812 may have some or all of the features discussed above; however, according to this embodiment, filter 812 has a decreasing inner diameter (ID) along the length thereof. Filter 812 may be positioned within, e.g., the stent body illustrated in FIG. 7.

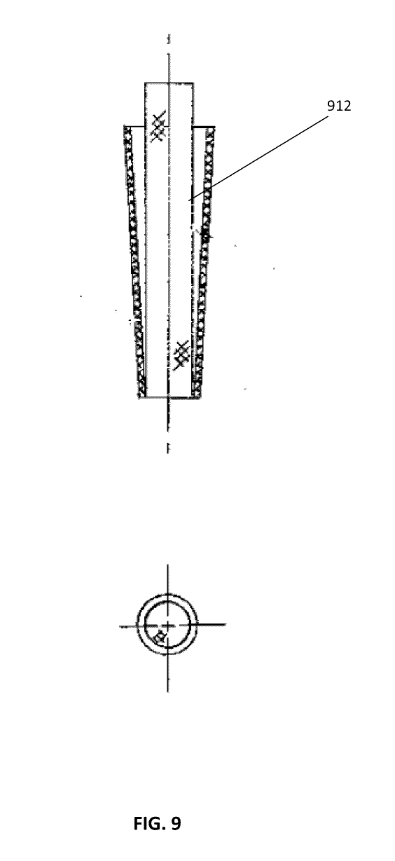

FIG. 9 illustrates a side view of a filter according to an alternative embodiment of the invention. Similar to the previous embodiment, filter 912 may have some or all of the features discussed above; however, according to this embodiment, filter 912 is tubular in configuration. In alternative embodiments, filter 912 may be an elongated rectangular tube. Filter 912 may be positioned within, e.g., the stent body illustrated in FIG. 7.

According to embodiments of the invention, the nasal stent may be collapsible from a first position (collapsed) to a second position (expanded). The first position may be when the nasal stent is collapsed on a delivery device before implanting the nasal stent into the nostril and nasal canal of a patient. Generally, this is accomplished by crimping the stent onto the delivery device and securing it in the first position with a shroud. When inserted into the nostril or surgically implanted, the nasal stent is in the first or collapsed position. Once fitted therein, the shroud is removed and expanded to the second position. The nasal stent may be balloon-expandable or self-expandable as known by one of ordinary skill in the art. Although a tubular configuration is illustrated in the drawings, it should be appreciated that other configurations, such as rectangular or oval, are within the scope of the invention to suit nostrils of various shapes and size.

An abluminal, luminal and/or combination thereof surface of a nasal stent as described may or may not be coated with appropriate therapeutic agents to suit a patient's symptoms. An example of this kind of stent is typically referred to as a drug-eluting stent (DES). In the case of a drug-eluting stent, the therapeutic agent may include, but is not limited to, an allergenic antigen-specific antibody, an anti-inflammatory, an antihistamine, a steroid, or a vaccine (e.g., the H1N1 vaccine). The antibodies used may be those in which their constant (or FC) regions are modified such that they are unable to bind to mast cells which otherwise results in a partial or complete inability to promote an immune response. More particularly, the therapeutic agent may be a glucocorticoid, leukotriene modifier or mast cell stabilizer. The therapeutic agent may or may not be combined with a controlled-release agent such as those known by one of ordinary skill in the art. Moreover, the nasal stent may provide for regulated breathing and purified environmental air before it is ingested into the lungs through the nasal cavity.

While certain exemplary embodiments have been described and shown in the accompanying drawings, it is to be understood that such embodiments are merely illustrative of and not restrictive on the broad invention, and that this invention is not to be limited to the specific constructions and arrangements shown and described, since various other modifications may occur to those ordinarily skilled in the art.

What is claimed is:

1. A nasal stent, comprising:
a collapsible body having a proximal end and a distal end, the collapsible body having a lumen therein; and
a filter mechanism positioned (i) within the collapsible body or (ii) outside of the collapsible body.

2. The nasal stent of claim 1 wherein the collapsible body tapers from the distal end to the proximal end.

3. The nasal stent of claim 2 wherein the filter mechanism is positioned within the collapsible body and tapers from a distal end to a proximal end thereof.

4. The nasal stent of claim 2 wherein the filter mechanism is tubular in configuration or is an elongated rectangular tube.

5. The nasal stent of claim 3 wherein the collapsible body comprises a mesh-like design from one of stainless steel, nickel-titanium, or a bioabsorbable or non-bioabsorbable polymer.

6. The nasal stent of claim 4 wherein the collapsible body comprises a mesh-like design from one of stainless steel, nickel-titanium, or a bioabsorbable or non-bioabsorbable polymer.

7. The nasal stent of claim 5 wherein an abluminal surface of the collapsible body is coated with a therapeutic agent.

8. The nasal stent of claim 6 wherein an abluminal surface of the collapsible body is coated with a therapeutic agent.

9. The nasal stent of claim 7 wherein the therapeutic agent is one of an allergenic antigen-specific antibody, an anti-inflammatory, an antihistamine, a steroid, or a vaccine.

10. The nasal stent of claim 8 wherein the therapeutic agent is one of an allergenic antigen-specific antibody, an anti-inflammatory, an antihistamine, a steroid, or a vaccine.

11. The nasal stent of claim 9 wherein the therapeutic agent is combined with a controlled-release agent.

12. The nasal stent of claim 10 wherein the therapeutic agent is combined with a controlled-release agent.

13. The nasal stent of claim 1 wherein the wherein the filter mechanism is positioned outside the collapsible body, the nasal stent further comprising:
a hollow insert positioned within the proximal end of the collapsible body;
a hollow reinforcing mechanism positioned within the hollow insert, and
a support engaged with the proximal portion of the body.

14. The nasal stent of claim 13 wherein the collapsible body comprises a mesh-like design from one of stainless steel, nickel-titanium, or a bioabsorbable or non-bioabsorbable polymer.

15. The nasal stent of claim 14 wherein an abluminal surface of the collapsible body is coated with a therapeutic agent.

16. The nasal stent of claim 15 wherein the therapeutic agent is one of an allergenic antigen-specific antibody, an anti-inflammatory, an antihistamine, a steroid, or a vaccine.

17. The nasal stent of claim 16 wherein the therapeutic agent is combined with a controlled-release agent.

18. The nasal stent of claim 1 wherein the filter mechanism has carbon media therein.
